(19) United States
(12) Patent Application Publication
(10) Pub. No.: US 2005/0031686 A1
(43) Pub. Date: Feb. 10, 2005

(54) Gastroresistant tablets for alimentary, dietetic and therapeutic use

(75) Inventors: Alessandro Seneci, Segrate (IT); Pia Alberico, Milano (IT)

Correspondence Address: DYKAS, SHAVER & NIPPER, LLP, P.O. BOX 877, 802 WEST BANNOCK STREET, SUITE 405, BOISE, ID, 83701, US

(73) Assignee: Truffini & Regge' Farmaceutici SRL, Milano (IT)

(21) Appl. No.: 10/898,664

(22) Filed: Jul. 23, 2004

Related U.S. Application Data
(63) Continuation-in-part of application No. 10/089,262, filed on Mar. 25, 2002.

(51) Int. Cl. A61K 9/48; A61K 9/22

(52) U.S. Cl. 424/468

(57) ABSTRACT

A novel formulation for oral use containing at least one active principle with a pharmaceutical, dietary or alimentary action, in combination with at least one fat in an amount of between 5 and 30%, relative to the weight of the formulation; this formulation allows the slow release, over time, of the active principle under physiological conditions which simulate the digestive processes which normally take place in the stomach.

### PRIORITY

This application claims the priority of and is a Continuation in Part of the utility application entitled Gastroresistant Tablets for Alimentary, Dietetic and Therapeutic Use, filed by Seneci et al. on Mar. 25, 2002 with application Ser. No. 10/089,262, the disclosure of which is incorporated herein.

### FIELD OF THE INVENTION

The present invention generally relates to gastro-resistant formulations, preferably tablets, for alimentary or dietary use, which are obtained by mixing the composition with fat in order to achieve a prolonged release of the active principles contained therein to the organism.

The preparation of the gastro-resistant formulations is usually carried out so as to allow the active principle to be released and absorbed in a more or less retarded manner at the intestine level; alternatively, the active principle may be released and absorbed only in part at the stomach level, thus allowing a second fraction of the active principle to be released and absorbed at the intestine level.

The known technique for preparing gastro-resistant formulations with retarded release is as follows:
A) Gastro-resistant formulations: these are tablets lined with gastro-resistant films, for example, ethylcellulose, cellulose acetophthalate, polyacrylates, gum lac and keratine; the lined tablets are then coated with sugar.
B) Layered formulations: they are prepared in the same manner as the gastro-resistant sugar coated pills. With regard to the coating of the tablets, a sprinkling powder such as starch or talcum, in which an active principle is dispersed using a water-soluble product such as gum arabic, agar-agar etc. as the adhesive, is attached in layers to the coated core in such a manner that the outermost layer, not the inner layer, of the tablet is dissolved in the stomach.
C) Capsules containing retarding agents are sugar cores in which the active principle is dispersed, followed by the application of a protective coating as in paragraph A).
D) Tablets in which retarding agents are dispersed in such a way that part of the active principle is present in the gastro-resistant retarding agents and part is present in the water-dispersible tablet.
E) Multi-layered tablets in which one or more layers contain dissolution retarding powders, such as cellulose derived gum lacs, so that the layers have different solubility.

In general, they are formulations whose retarding effect is based on the use of excipients and/or adjuvants foreign to the mammalian organism, in particular of humans, which formulations are intended to maximize the absorption of the active principle without taking into consideration the normal physiological digestive processes.

However, the use of such substances is usually not very desirable, in particular in the case of dietary formulations and/or in the case of food additives, which are intended to instead achieve absorption of the active principle according to a kinetic profile, which is as close as possible to the normal human digestive processes.

The recourse to “natural” absorption profiles is anyway desirable, even in the case of therapeutic formulations. One example is in all of the classes of patients who would be harmed by administering them non-“physiological” excipients and/or adjuvants. Some examples of these classes are pregnant women, very young children, allergic subjects, etc.

Now, according to the subject matter of the present invention, a novel fomulation with retarded release has been found, said formulation allowing the active principles to be absorbed utilizing the physiological digestive activity, i.e. imitating what happens with food ingested in the usual manner. The present invention relates to a formulation in tablet form for oral use, containing at least one active principle with a pharmaceutical, dietary or alimentary action in combination with at least one fat as the vehicle in an amount of between 5 and 30%, relative to the weight of the formulation; preferably, such fat is present in an amount of between 20 and 30%, relative to the weight of the formulation.

The fatty acids contained in the fat that can be used for the purposes of the present invention are normally selected from those containing hydrogenated and non-hydrogenated fatty acids, either of synthetic or natural origin, having a chain comprising between 3 and 20 carbon atoms, preferably between 14 and 18 carbon atoms, and mixtures thereof.

A non-limiting list of such acids comprises, for example, palmitic acid, stearic acid, myristic acid, lauric acid, caprylic acid, capric acid, etc. From a practical point of view, the fats can normally be selected from among cocoa butter, hydrogenated palm oil, hydrogenated vegetable fats such as peanut butter, animal fats such as lard, butter, or bacon fat separately or in a mixture thereof.

As indicated above, the active principles that can be used for the purposes of the present invention may have both a therapeutic and a dietary or alimentary action. The active principles with a therapeutic action may be selected from among non-steroid anti-inflammatory drugs (NSAID) and steroid anti-inflammatory drugs. Steroid anti-inflammatory drugs may be selected from tranquilizers, sleeping pills, anti-hypertensive, anti-histaminic and anti-asthmatic drugs. Non-steroid anti-inflammatory drugs in turn may be selected from among ibuprofen, naproxen, ketoprofen, indomethacin, acetylsalicylic acid, mefenamic acid, flufenamic acid, etc. The active principles with a dietary action may be selected from the group consisting of lactic acid micro organisms, beer yeasts, either as such or containing living cells, vitamins, minerals, amino acids, vegetable extracts and derivatives thereof.

In the formulation according to the present invention, the active principle or principles that may be used as such or in the form of esters or physiologically acceptable salts are mixed directly with at least one fat without the addition of any excipients and/or adjuvants, the active principle or principles making up 70-95% by weight, preferably 75-90% by weight, of the formulation (at least one active principle and at least one fat make up 100%, by weight, of the formulation).

In order to determine the release activity, over time, of an active principle contained in a formulation according to the present invention (the qualitative and quantitative composition of which is given in Example 1), the dissolution test described in Farmacopea Ufficiale Italiana (Official Italian Pharmacopeia) was carried out. The results of said test are shown in Table I.

[image]

This dissolution test demonstrates the slow release, over time, of an active principle under pysiological conditions, which simulates the digestive processes that normally take place in the stomach.

The present invention is particularly suitable for the production of BIO-certified gastro-resistant tablets, provided that fats derived from biological cultivations and farms in accordance with current regulations are used.

The present invention furthermore relates to the process for the preparation of the formulations according to the present invention.

The process comprises mixing at least one active principle with at least one fat in the melted state in the weight proportions defined above. The blend thus obtained is cooled to 5 to 20° C., preferably to 10° C. to 12° C., and then granulated, for example using an oscillating granulator of the Manesty type equipped with a perforated stainless steel plate having holes with a diameter of 1 to 4 mm, preferably 1 to 2 mm.

The granules thus obtained are compressed with a rotary tablet-compressing machine equipped with suitable punches. It is thus possible to obtain tablets of suitable weight.

In particular, the present invention is highly suitable for the preparation of layered tablets obtained with a suitable tablet-compressing machine such as a Manesty BB3B.

The process consists of compressing a layer obtained according to the prior art using one or more active principles mixed with known water-soluble or water-dispersible excipients, and one layer obtained according to the present invention. If desired, it is also possible to use more than two layers with different degrees of solubility. The layer obtained according to the prior art (i.e. the conventional layer) is preferably a fast release formulation.

The examples that follow are given in order to better describe the present invention without, however, limiting its scope.

### EXAMPLE 1

1000 tablets are prepared, being formed by a fast-dissolving layer (Layer A) obtained by kneading, in a Z-type kneader, the following components together with 10% strength Klucel/water:
proline (100 g)
lysine (100 g)
cystine (100 g)
sodium carboxymethylcellulose (20 g)

The blend thus obtained is dried for 12 hours at 40° C. in a drying cabinet; the resulting mixture is granulated in a Manesty granulator equipped with a perforated stainless steel plate having holes with a 2 mm diameter, giving a yield of 321.8 g.

The granules thus obtained are mixed in a rotating-screw mixer (SAGA) with:
red lake N° 40 all lake (0.25 g)
vitamin A 5,000,000 IU/g (800 μg/cpr+30%) (2.31 g)
vitamin E 50% SD (16 mg/cpr+20%) (12.8 g)
vitamin C granules (49.5 g)
magnesium stearate (5 g)
copper gluconate Cu 14% (1.2 mg/cpr+5%) (6 g)
zinc gluconate Zn 13.4% (10 mg/cpr+5%) (52.2 g)
selenium-containing yeast 2,000 μg/g (0.055 μg/cpr+5%) (19 g)
glutathione on yeast (25 mg/cpr+20%) (15 g)
rapidly disintegrating PVP (20 g)
potato starch (10 g)
silica gel (3 g)
maltodextrin (5 g)
microcrystalline cellulose (2 g)
water (0.5 g)
giving a total yield of 524.36 g

A second mixture is prepared and used to form the slow-dissolving layer (LAYER B) thus obtained:
lyophilized blueberry (15 g)
microcrystalline cellulose (50 g)
titanium dioxide (10 g)
nucleic acids (50 g)
blueberry extract 25% (50 g)
copper gluconate (1.5 g)
zinc gluconate (12.3 g)
copper gluconate (1.5 g)
zinc gluconate (13.8 g)
selenium-containing yeast (9.5 g)
glutathione on yeast (15 g)
vitamin A 500,000 IU/g (4.63 g)
vitamin E 50% SD (25.6 g), vitamin C EC 97% (99 g)

All of these components are mixed and kneaded in a Z-type kneader together with melted hydrogenated palm oil (50 g).

The blend obtained is cooled to 12° C. and granulated in an oscillating granulator equipped with a stainless steel plate having holes with a 2 mm diameter, giving a total yield of 408 g.

The two mixtures thus obtained can be compressed with an oval punch using a double-layered tablet-compressing machine (MANESTY BB3B) producing oval tablets with a weight of 0.932 g, in which the first layer weighing 0.524 g is fast-dissolving and the second layer weighing 0.408 g is gastro-resistant and slow dissolving.

### EXAMPLE 2

Example 1 is repeated except that the following components are used:

Layer A (FAST-DISSOLVING)

folic acid 98% (0.3 mg/cpr+20%) (0.12 g)
vitamin B6 33.1/3 (1.5 mg+20%) (1.8 g)
beta carotene 20% (4 mg/cpr+10%) (7.4 g)
vitamin E 50% SD (116 mg/cpr) (12.8 g)
vitamin C EC 97 (120 mg/cpr+20%) (49.5 g)
copper gluconate Cu 14% (1.2 mg/cpr) (6 g)
zinc gluconate Zn 13.4% (10 mg/cpr) (52.3 g)
selenium-containing yeast 2000 μg/g (55 μg/cpr) (19.3 g)
lactose CD (150 g)
microcrystalline cellulose (30 g)
water (4 g)
potato starch (30 g)
rapidly disintegrating PVP (Kollidon CL) (10 g)
silicagel (10 g)
maltodextrin (8 g)
giving a total of 391.22 g:
   Layer B (SLOW-DISSOLVING)
sulfomucopolysaccharides (25 g)
Gingko biloba (30 g)
copper gluconate Cu 14% (3 g)
zinc gluconate Zn 13.4% (26.2 g)
selenium-containing yeast 2,000 μg/g (9.7 g)
microcrystalline cellulose (50 g)
red iron oxide (5 g)
folic acid (0.24 g)
vitamin B6 33.1/3% (3.6 g)
vitamin E 50% (25.6 g)
vitamin C EC 97% (99 g)
beta carotene 20% (14.8 g)
melted hydrogenated palm oil (72 g)
silica gel (0.5%)
giving a total of 0.358 g

Double-layered tablets weighing 0.749 g are prepared, the first layer of which weighing 0.391 g is fast dissolving and the second one weighing 0.358 g is slow dissolving.

The tablets can then be coated with a solution of 10% strength Klucel/water.

### EXAMPLE 3

Example 1 is repeated except that the following components are used:

Layer A (FAST-DISSOLVING)

| | |
|---|---|
| acetylsalicylic acid | 0.3 g |
| hydrogenated palm oil | 0.1 g |
| lactose | 0.2 g |

Layer B (SLOW-DISSOLVING)

| | |
|---|---|
| acetylsalicylic acid | 0.2 g |
| lactose | 0.1 g |
| magnesium stearate | 0.01 g |
| pre-dried corn starch | 0.1 g |

### EXAMPLE 4

1.2 grams retarded release tablets can be obtained by compressing a granulate obtained according to the previous examples and having the following composition:

| | |
|---|---|
| Vitamin C | 0.2% |
| Vitamin PP | 0.07% |
| Calcium panthotenate | 0.0243% |
| Vitamin B6 | 0.022% |
| Vitamin B1 | 0.016% |
| Vitamin B2 | 0.017% |
| Vitamin B12 | 0.0004% |
| Folic acid | 0.0007% |
| Biotin | 0.0006% |
| Proteic hydrolizate | 80.00% |
| Hydrogenated palm oil | 19.649% |
| Total | 100.00% |

### EXAMPLE 5

1.2 grams retarded release tablets can be obtained by compressing a granulate obtained according to the previous examples and having the following composition:

| | |
|---|---|
| Vitamin C | 0.2% |
| Vitamin PP | 0.07% |
| Calcium panthotenate | 0.0243% |
| Vitamin B6 | 0.022% |
| Vitamin B1 | 0.016% |
| Vitamin B2 | 0.017% |
| Vitamin B12 | 0.0004% |
| Folic acid | 0.0007% |
| Biotin | 0.0006% |
| Inulin | 80.00% |
| Hydrogenated palm oil | 19.649% |
| Total | 100.00% |

1. A retarded release formulation for oral use in tablet form, consisting of at least one active principle with a pharmaceutical, dietary or alimentary action in an amount of between 70 and 95% and at least one fat in an amount of between 5 and 30%, relative to the weight of the formulation.

2. The formulation according to claim 1, wherein at least one fat is an amount of between 10 and 20%, relative to the weight of the formulation.

3. The formulation according to claim 1, wherein at least one fat contains hydrogenated and non-hydrogenated fatty acids, either of synthetic or natural origin, having a chain comprising between 3 and 20 carbon atoms.

4. The formulation according to claim 1, wherein at least one fat contains hydrogenated and non-hydrogenated fatty acids, either of synthetic or natural origin, having a chain comprising between 14 and 18 carbon atoms, or mixtures thereof.

5. The formulation according to claim 1, wherein at least one fat is selected from cocoa butter, hydrogenated palm oil, hydrogenated vegetable fats such as peanut butter, animal fats such as lard, butter, bacon fat.

6. The formulation according to claim 1, wherein at least one active principle is present in an amount of 75-90%, relative to the weight of the formulation.

7. The formulation according to claim 1, wherein at least one active principle with a therapeutic action is selected from among non-steroid and steroid anti-inflammatory drugs, tranquilizers, sleeping pills, anti-hypertensive, anti-histaminic and anti-asthmatic drugs and in that at least one active principle with a dietary or alimentary action is selected from the group consisting of lactic acid microorganisms, beer yeasts, either as such or containing living cells, vitamins, minerals, amino acids, vegetable extracts, and derivatives thereof.

8. A layered tablet consisting of at least one first layer which is a retarded release formulation according to claim 1 and at least one conventional second layer.

9. A tablet according to claim 8, wherein at least one conventional second layer is a fast release formulation.

10. Process for the preparation of a formulation according to claim 1 in which:
a) at least one active principle is mixed with said at least one fat in the melted state in the defined weight proportions;
b) the blend thus obtained is cooled to 5-20° C. and then granulated using a granulator having holes with a diameter of between 1 and 4 mm; and
c) the granules thus obtained are then compressed.

11. Process according to claim 10 wherein the blend is cooled to 10° C.-12° C.

12. Process according to claim 10 wherein the blend is granulated using a granulator having holes with a diameter of between 1 and 2 mm.
